(19) United States
(12) Patent Application Publication
(10) Pub. No.: US 2003/0186437 A1
(43) Pub. Date: Oct. 2, 2003

(54) Use of RAIDD to control cell differentiation

(75) Inventors: Anthony John Clark, Midlothian (GB); Ricardo Felmer, Midlothian (GB); Simon Horvat, Midlothian (GB)

Correspondence Address: HALE AND DORR, LLP, 60 STATE STREET, BOSTON, MA, 02109

(73) Assignee: ROSLIN INSTITUTE, Roslin (GB)

(21) Appl. No.: 10/114,017

(22) Filed: Mar. 29, 2002

(51) Int. Cl. C12N 5/08; C12N 15/85

(52) U.S. Cl. 435/366; 435/455; 435/368; 435/370; 435/372

(57) ABSTRACT

A method of controlling the differentiation of a cell is provided which comprises modulation of the expression of the gene Raidd in the cell. The method provides a means for preparing or enriching a population of stem cells through modulation of Raidd expression.

The present invention relates to a means for controlling the differentiation of cells and enrichment of stem cell or progenitor cell populations.

A cell is considered as being “differentiated” with respect to an undifferentiated cell when it acquires distinguishing characteristics of morphology or phenotype. Cell differentiation is a process affecting cells in the embryo, (including the foetus) and the adult organism. Stem cells or progenitor cell populations are found in all stages of development of the animal body through to (and including) the adult. An undifferentiated or partly differentiated cell has developmental plasticity in that it has the capability to develop and differentiate further.

In a developing embryo, the cells in the embryo are affected by the influence of various factors that commit the cell or cells to differentiate into a defined cell type. At early stages of development, embryonic stem (ES) cells can be derived from the pluripotent inner cell mass (ICM) of the blastocyst stage embryo in certain species notably mice and humans. ES cells are similar to embryonic germ (EG) cells which are derived from the primordial germ cells (PGCs) of the developing embryo. A key feature of stem cells is that they are capable of undergoing both self-renewal and differentiation. A number of growth factors and biochemical signals have been identified that control development of ES and EG cells and regulate this balance between self renewal and differentiation. Initial differentiation of cell types appears to be concerned with the positional location of the cells within the embryo for some cell lineages. Subsequent differentiation relates to the formation of specific cell types leading to the development of recognisable tissues and organs. Both ES and EG cells are thought to be pluripotent in that they have the developmental potential to differentiate into any somatic cell type and germ cells.

In all stages of development and in the adult (including new-born), there are progenitor stem cell populations that undergo differentiation ultimately to become defined cell-types. Maintenance of adult tissues requires the presence of a self-renewing renewing population of stem cells to provide new cells to replace damaged or dead cells. For example, haematopoietic stem cells (HSCs) that undergo differentiation to become the cells of the haematopoeitic cell lineage, including erythrocytes, leukocytes etc; or mesenchymal stem cells or skeletal stem cells (SSCs) that can differentiate into cells required to form bone, cartilage, adipocytes and haematopoiesis-supporting stroma (Friedenstein et al J. Embryol. Exp. Morphol. 16 381-390 (1966); Bianco et al J. Clin. Invest. 105 1663-1668 (2000); Bianco et al Stem Cells 19 180-192 (2001)). Haematopoietic stem cells (HSCs) as well as SSCs have been shown to have the potential for myogenesis and cardiomyogenesis (Kocher et al Nature Medicine 7 430-436 (2001); Ferrari et al Science 279 1528-1530 (1998); Orlic et al Nature 410 701-705 (2001)). It seems that SSCs can give rise to neural cells, and that HSCs can develop into liver cells (reviewed by Bianco, P. & Robey, P. G. Nature 414 118-121 (2001)). Many adult derived stem cell populations are multipotent—that is they are able to differentiate into a number of different cell type. Unlike ES or EG cells they are not pluripotent as they do not have the developmental potential to form all the somatic cell types or germ cells.

The discovery of stem cell populations in both embryos and adults raises the prospect of cell-based therapies for treatment of diseases using progenitor cells isolated from the adult. Such therapies will require control over the differentiation of the progenitor cell populations. However, means for isolating and enriching such cells have not been fully identified and would benefit from improvements.

The development of cell nucleus replacement (CNR) technology (nuclear transfer or cloning) has also opened up the possibility of obtaining defined sources of pluripotent embryonic stem cells and/or embryonic germ cells from which to derive specific cell types for use in medicine.

Stem cells or progenitor cells (however obtained) may be induced to differentiate into desired cell types, for example myocardiocytes or neural cell types, by addition of the appropriate growth factors or biochemical factors. However, in certain circumstances it may also be desirable to inhibit further differentiation so as to permit other techniques to be performed prior to final cell differentiation.

RAIDD (RIP-associated ICH-1 homologous protein with a death domain) is an adaptor molecule that contains an amino-terminal CARD (Caspase Recruitment Domain) region and a carboxy-terminal “death domain” (Duan, H. & Dixit, V. M., Nature 385 86-9 (1997). The carboxy-terminal domain interacts with the homologous death domain region in RIP, a serine/threonine kinase component of the TNFRI signalling complex, and the Caspase-2 (ICH-1) molecule associates with the homologous amino-terminal CARD domain of RAIDD (Cohen, G. M. Biochem J. 326 1-16 (1997)). The human death adaptor molecule RAIDD which shares a very high homology with the mouse Raidd gene (Horvat. S, & Medrano, J. F., Genomics 54 159-64 (1998)), was tested in MCF-7 cell lines (a human breast carcinoma cell) to address the function of RAIDD in apoptosis. MCF-7 cells transiently transfected with a RAIDD construct underwent apoptosis, which was inhibited by the inclusion of several known inhibitors of apoptosis, thus confirming the role of this gene in the apoptotic pathway (Duan, H. & Dixit, V. M., Nature 385 86-9 (1997)).

It has now been surprisingly found that the Raidd gene codes for a protein which can also control or inhibit differentiation in cells. Modulation of the expression of Raidd will enable control of cellular differentiation to be realised.

According to a first aspect of the invention, there is provided a method of controlling the differentiation of a cell, the method comprising modulating the expression of Raidd in the cell.

The process of differentiation as used in the context of the present invention includes the developmental processes that occur in the cells of an animal during determination and terminal differentiation. A cell is “determined” if it has undergone a self-perpetuating change of internal character that distinguishes it and its progeny from other cells in the animal and commits them to a specialised course of development. A cell that is “determined” may not show any outward signs of “differentiation” in terms of gross morphology. The term “differentiation” also refers to cells having overt cell specialisation. In many cases, determination and differentiation may occur simultaneously and in other cases differentiation can occur without any prior determination. Undifferentiated or progenitor cell populations (including stem cells), can be found in animals at all stages of development, from embryo, through foetus, up to an including the adult.

The cell in which differentiation is controlled may be at any stage in the process of cell differentiation. It may be fully undifferentiated, or partly differentiated but not yet exhibiting its final morphological characteristics (and so not terminally “differentiated”).

Progenitor stem cell populations that can give rise to subsequent cell types, include but are not limited to haematopoietic stem cells (HSCs), mesenchymal stem cells or skeletal stem cells (SSCs), muscle stem cells, neural stem cells, umbilical cord stem cells, embryonic stem (ES) cells, embryonic germ (EG) cells and pluripotent stem cells. Pluripotent stem cells have the potential to develop into any cell in the body and at present such stem cells can be isolated from ES cells, EG cells, or from embryonal carcinoma (EC) cells. EC cells are the stem cells of teratocarcinomas.

Stem cell or progenitor cells or cell populations may be obtained from any convenient tissue or organ source by means of isolation in vitro, extraction from an in vivo subject, or from a recent post-mortem subject. In developing embryos, the source of ES cells is the inner cell mass of the blastocyst. Subsequently, through embryonic development into the adult, other stem cells or progenitor cells may be found in the appropriate tissue of interest, for example in the bone marrow for haematopoietic stem cells, in neural tissue for neural stem cells. Stem cell niches have also been observed in the mammalian testis (germline stem cells or GSCs), Drosophila testis (GSCs and somatic stem cells), Drosophila ovariole (GSCs), Caenorhabditis elegans (candidate niche identified), mammalian epithelial skin (matrix stem cells for maintenance of hair follicles, sebaceous glands, sweat glands etc.), Drosophila follicle cells, endodermal gut crypts (reviewed by Spradling et al Nature 414 98-104 (2001)). Stem cells can also be generated from embryos created by nuclear transfer procedures at the blastocyst stage, or from ES-somatic cell or EG-somatic cell hybrids (reviewed by Surani, M. A., Nature 414 122-128 (2001)).

Controlling differentiation includes complete inhibition of differentiation of the cell, as well as partial inhibition sufficient to prevent morphological and/or biochemical changes associated with differentiation

Modulation of the expression of Raidd in the cell, includes overexpression (that is to say at levels of expression above the normal for the cell type concerned), as well as underexpresion (that is to say inhibition or partial inhibition of gene expression of Raidd to levels below the normal for the cell type concerned). The term “modulate” when used herein in reference to expression or activity of a RAIDD or a RAIDD-related polypeptide refers to the upregulation or downregulation of the expression or activity of RAIDD or a RAIDD-related polypeptide. Based on the present disclosure, such modulation can be determined by assays known to those of skill in the art or described herein.

Overexpression of Raidd means expression of the Raidd protein at levels above the normal rate for expression in the cell concerned. In many cases, this can be accomplished by the use of inducible regulatory systems including but not limited to inducing agents, responsive promoter sequences and enhancer elements, locus control regions and trans activating factors and a combination of some or all of these component. Examples include the Tet repressor based system (Baron, U. & Bujard, H. Methods Enzymol. 327 401-421 (2000)); the CYP1A1 promoter (Campbell et al J. Cell Sci. 109 2619-2625 (1996); and the ecdysone responsive regulatory system (No et al Proc. Nat'l. Acad. Sci. USA 93(8) 3346-3351 (1996)); a tissue specific promoter (for example AP2 expressed specifically in the adipocyte lineage of cells (Graves et al J. Cell. Biochem. 49(3) 29-224 (1992)); the nestin enhancer element expressed specifically in neuronal lineages of cells (Lothian, C & Lendahl, U. Eur. J. Neurosci. 9(3) 452-462 (1997)); the pdx gene expressed specifically in pancreatic b-cell lineages (Gannon et al Dev. Biol. 238 (1) 185-201 (2001)); the albumin enhancer and promoter elements expressed specifically in the liver cells Pinkert et al Genes Dev. 1(3) 268-276 (1987)); and the β-globin LCR and promoter elements which target expression specifically to erythroid cells (Needdham et al Nucleic Acids Res. 20(5) 997-1003 (1992)); or other suitable heterologous regulatory elements effective for driving expression of Raidd. Overexpression of Raidd may be constitutive in the cell.

The promoter may be introduced into the genome of the cell to activate expression of the native Raidd gene, or it may be part of a nucleic acid construct comprising the promoter and a non-native Raidd gene which is introduced into the genome of the cell.

Alternative means of achieving, higher than normal levels of expression could include introducing the Raidd gene through homologous recombination so that it was expressed from endogenous regulatory elements (for example see Mountford et al Proc. Nat'l Acad. Sci. USA 91(10) 4303-4307 (1994)) such that the gene was constitutively expressed in the cell.

Modulation of expression of Raidd may also be achieved by inhibiting or preventing expression of Raidd. This may conveniently be accomplished by standard gene “knock-out” technology (for example see, Thomas, K. R. & Capecchi, M. R. Nature 324 34-38 (1986)); or other more recent technologies such as RNAi (Elbashir et al Nature 411 494-498 (2001)) which may ablate gene expression.

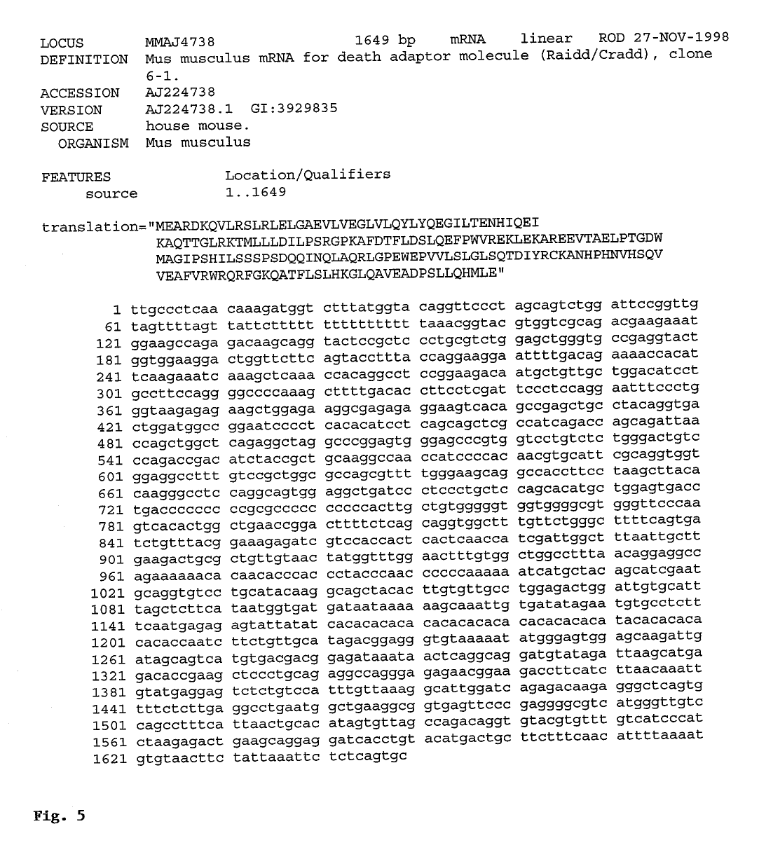

In the present invention, the Raidd gene is a nucleic acid sequence encoding the RAIDD protein (RIP-associated ICH-1 homologous protein with a death domain) (Duan, H. & Dixit, V. M., Nature 385 86-9 (1997)) as shown in FIG. 5 from Horvat S., & Medrano, J. F. in Genomics 54 (1) 159-164 (1998). The nucleic acid sequences that can be used in accordance with the present invention includes all fragments of Raidd encoding functional RAIDD protein, and nucleic acid sequences complementary or homologous to Raidd.

The skilled person can select appropriate primer sequences based on standard molecular biological techniques (Sambrook et al Molecular Cloning: A Laboratory Manual, Second edition (1989)). Such nucleic acid sequences may be used directly, or more commonly they will be used to design appropriate primers for use in PCR based procedures.

A nucleic acid sequence which is complementary to a nucleic acid sequence useful in a method of the present invention is a sequence which hybridises to such a sequence under stringent conditions, or a nucleic acid sequence which is homologous to or would hybridise under stringent conditions to such a sequence but for the degeneracy of the genetic code, or an oligonucleotide sequence specific for any such sequence. The nucleic acid sequences include oligonucleotides composed of nucleotides and also those composed of peptide nucleic acids. Where the nucleic sequence is based on a fragment of the gene encoding RAIDD, the fragment may be at least any ten consecutive nucleotides from the gene, or for example an oligonucleotide composed of from 20, 30, 40, or 50 nucleotides.

Stringent conditions of hybridisation may be characterised by low salt concentrations or high temperature conditions. For example, highly stringent conditions can be defined as being hybridisation to DNA bound to a solid support in 0.5M NaHPO₄, 7% sodium dodecyl sulfate (SDS), 1 mM EDTA at 65° C., and washing in 0.1×SSC/0.1% SDS at 68° C. (Ausubel et al eds. “Current Protocols in Molecular Biology” 1, page 2.10.3, published by Green Publishing Associates, Inc. and John Wiley & Sons, Inc., New York, (1989)). In some circumstances less stringent conditions may be required. As used in the present application, moderately stringent conditions can be defined as comprising washing in 0.2×SSC/0.1% SDS at 42° C. (Ausubel et al (1989) supra). Hybridisation can also be made more stringent by the addition of increasing amounts of formamide to destabilise the hybrid nucleic acid duplex. Thus particular hybridisation conditions can readily be manipulated, and will generally be selected according to the desired results. In general, convenient hybridisation temperatures in the presence of 50% formamide are 42° C. for a probe which is 95 to 100% homologous to the target DNA, 37° C. for 90 to 95% homology, and 32° C. for 70 to 90% homology.

Uses and methods in accordance with the present invention therefore extend to the use of nucleic acid sequences encoding RAIDD analogs, homologs, orthologs, fusion proteins, related polypeptides, derivatives, fragments, or isoforms thereof

The term “analog” as used herein refers to a polypeptide that possesses a similar or identical function as RAIDD but need not necessarily comprise an amino acid sequence that is similar or identical to the amino acid sequence of RAIDD, or possess a structure that is similar or identical to that of RAIDD. As used herein, an amino acid sequence of a polypeptide is “similar” to that of RAIDD if it satisfies at least one of the following criteria: (a) the polypeptide has an amino acid sequence that is at least 30% (more preferably, at least 35%, at least 40%, at least 45%, at least 50%, at least 55%, at least 60%, at least 65%, at least 70%, at least 75%, at least 80%, at least 85%, at least 90%, at least 95% or at least 99%) identical to the amino acid sequence of RAIDD; (b) the polypeptide is encoded by a nucleotide sequence that hybridizes under stringent conditions to a nucleotide sequence encoding at least 5 amino acid residues (more preferably, at least 10 amino acid residues, at least 15 amino acid residues, at least 20 amino acid residues, at least 25 amino acid residues, at least 40 amino acid residues, at least 50 amino acid residues, at least 60 amino residues, at least 70 amino acid residues, at least 80 amino acid residues, at least 90 amino acid residues, at least 100 amino acid residues, at least 125 amino acid residues, or at least 150 amino acid residues) of RAIDD; or (c) the polypeptide is encoded by a nucleotide sequence that is at least 30% (more preferably, at least 35%, at least 40%, at least 45%, at least 50%, at least 55%, at least 60%, at least 65%, at least 70%, at least 75%, at least 80%, at least 85%, at least 90%, at least 95% or at least 99%) identical to the nucleotide sequence encoding RIADD. As used herein, a polypeptide with “similar structure” to that of RAIDD refers to a polypeptide that has a similar secondary, tertiary or quarternary structure as that of RAIDD. The structure of a polypeptide can determined by methods known to those skilled in the art, including but not limited to, X-ray crystallography, nuclear magnetic resonance, and crystallographic electron microscopy.

The term “RAIDD fusion protein” as used herein refers to a polypeptide that comprises (i) an amino acid sequence of RAIDD, a RAIDD fragment, a RAIDD-related polypeptide or a fragment of a RAIDD-related polypeptide and (ii) an amino acid sequence of a heterologous polypeptide (i.e., a non-RAIDD, non-RAIDD fragment or non-RAIDD-related polypeptide).

The term “RAIDD homolog” as used herein refers to a polypeptide that comprises an amino acid sequence similar to that of RAIDD but does not necessarily possess a similar or identical function as RAIDD.

The term “RAIDD ortholog” as used herein refers to a non-human polypeptide that (i) comprises an amino acid sequence similar to that of RAIDD and (ii) possesses a similar or identical function to that of RAIDD.

The term “RAIDD-related polypeptide” as used herein refers to a RAIDD homolog, a RAIDD analog, an isoform of RAIDD, a RAIDD ortholog, or any combination thereof.

The term “derivative” as used herein refers to a polypeptide that comprises an amino acid sequence of a second polypeptide which has been altered by the introduction of amino acid residue substitutions, deletions or additions. The derivative polypeptide possess a similar or identical function as the second polypeptide.

The term “fragment” as used herein refers to a peptide or polypeptide comprising an amino acid sequence of at least 5 amino acid residues (preferably, at least 10 amino acid residues, at least 15 amino acid residues, at least 20 amino acid residues, at least 25 amino acid residues, at least 40 amino acid residues, at least 50 amino acid residues, at least 60 amino residues, at least 70 amino acid residues, at least 80 amino acid residues, at least 90 amino acid residues, at least 100 amino acid residues, at least 125 amino acid residues, at least 150 amino acid residues, at least 175 amino acid residues, at least 200 amino acid residues, or at least 250 amino acid residues) of the amino acid sequence of a second polypeptide. The fragment of RAIDD may or may not possess a functional activity of the second polypeptide.

The term “isoform” as used herein refers to variants of a polypeptide that are encoded by the same gene, but that differ in their pI or MW, or both. Such isoforms can differ in their amino acid composition (e.g. as a result of alternative splicing or limited proteolysis) and in addition, or in the alternative, may arise from differential post-translational modification (e.g., glycosylation, acylation, phosphorylation). As used herein, the term “isoform” also refers to a protein that exists in only a single form, i.e., it is not expressed as several variants.

The percent identity of two amino acid sequences or of two nucleic acid sequences is determined by aligning the sequences for optimal comparison purposes (e.g., gaps can be introduced in the first sequence for best alignment with the sequence) and comparing the amino acid residues or nucleotides at corresponding positions. The “best alignment” is an alignment of two sequences which results in the highest percent identity. The percent identity is determined by the number of identical amino acid residues or nucleotides in the sequences being compared (i.e., % identity=# of identical positions/total # of positions×100).

The determination of percent identity between two sequences can be accomplished using a mathematical algorithm known to those of skill in the art. An example of a mathematical algorithm for comparing two sequences is the algorithm of Karlin and Altschul Proc. Natl. Acad. Sci. USA (1990) 87:2264-2268, modified as in Karlin and Altschul (1993) Proc. Natl. Acad. Sci. USA 90:5873-5877. The NBLAST and XBLAST programs of Altschul et al, J. Mol. Biol. (1990) 215:403-410 have incorporated such an algorithm. BLAST nucleotide searches can be performed with the NBLAST program, score=100, wordlength=12 to obtain nucleotide sequences homologous to a nucleic acid molecules of the invention. BLAST protein searches can be performed with the XBLAST program, score=50, wordlength=3 to obtain amino acid sequences homologous to a protein molecules of the invention. To obtain gapped alignments for comparison purposes, Gapped BLAST can be utilized as described in Altschul et al, Nucleic Acids Res. (1997) 25:3389-3402. Alternatively, PSI-Blast can be used to perform an iterated search which detects distant relationships between molecules (Id.). When utilizing BLAST, Gapped BLAST, and PSI-Blast programs, the default parameters of the respective programs (e.g., XBLAST and NBLAST) can be used. See http://www.ncbi.nlm.nih.gov.

Another example of a mathematical algorithm utilized for the comparison of sequences is the algorithm of Myers and Miller, CABIOS (1989). The ALIGN program (version 2.0) which is part of the GCG sequence alignment software package has incorporated such an algorithm. Other algorithms for sequence analysis known in the art include ADVANCE and ADAM as described in Torellis and Robotti Comput. Appl. Biosci. (1994) 10:3-5; and FASTA described in Pearson and Lipman Proc. Natl. Acad. Sci. USA (1988) 85:2444-8. Within FASTA, ktup is a control option that sets the sensitivity and speed of the search.

In the present invention, the RAIDD molecule may be that coded for by the native Raidd gene, although it is envisaged that alternative synthetic forms of the molecule could be made by substitution of one or more amino acids in the molecule. The invention therefore extends to the use of a molecule having RAIDD activity. The skilled person is aware that various amino acids have similar properties. One or more such amino acids of a substance can often be substituted by one or more other such amino acids without eliminating a desired activity of that substance. Thus the amino acids glycine, alanine, valine, leucine and isoleucine can often be substituted for one another (amino acids having aliphatic side chains). Of these possible substitutions it is preferred that glycine and alanine are used to substitute for one another (since they have relatively short side chains) and that valine, leucine and isoleucine are used to substitute for one another (since they have larger aliphatic side chains which are hydrophobic). Other amino acids which can often be substituted for one another include: phenylalanine, tyrosine and tryptophan (amino acids having aromatic side chains); lysine, arginine and histidine (amino acids having basic side chains); aspartate and glutamate (amino acids having acidic side chains); asparagine and glutamine (amino acids having amide side chains); and cysteine and methionine (amino acids having sulphur containing side chains). Substitutions of this nature are often referred to as “conservative” or “semi-conservative” amino acid substitutions.

Amino acid deletions or insertions may also be made relative to the amino acid sequence of RAIDD. Thus, for example, amino acids which do not have a substantial effect on the activity of RAIDD, or at least which do not eliminate such activity, may be deleted. Amino acid insertions relative to the sequence of RAIDD can also be made. This may be done to alter the properties of a substance of the present invention (e.g. to assist in identification, purification or expression, where the protein is obtained from a recombinant source, including a fusion protein. Such amino acid changes relative to the sequence of RAIDD from a recombinant source can be made using any suitable technique e.g. by using site-directed mutagenesis. The RAIDD molecule may, of course, be prepared by standard chemical synthetic techniques, e.g. solid phase peptide synthesis, or by available biochemical techniques, e.g. expression of Raidd in a suitable host cell system.

It should be appreciated that amino acid substitutions or insertions within the scope of the present invention can be made using naturally occurring or non-naturally occurring amino acids. Whether or not natural or synthetic amino acids are used, it is preferred that only L-amino acids are present.

Whatever amino acid changes are made (whether by means of substitution, insertion or deletion), preferred polypeptides of the present invention have at least 50% sequence identity with a polypeptide as defined in a) above more preferably the degree of sequence identity is at least 75%. Sequence identities of at least 90% or at least 95% are most preferred.

The degree of amino acid sequence identity can be calculated using a program such as “bestfit” (Smith and Waterman, Advances in Applied Mathematics, 482-489 (1981)) to find the best segment of similarity between any two sequences. The alignment is based on maximising the score achieved using a matrix of amino acid similarities, such as that described by Schwarz and Dayhof (1979) Atlas of Protein Sequence and Structure, Dayhof, M. O., Ed pp 353-358. Where high degrees of sequence identity are present there will be relatively few differences in amino acid sequence.

Methods in accordance with this aspect of the invention may therefore additionally comprise a step of transfecting the cell with a nucleic acid construct comprising a promoter and a nucleic acid sequence encoding a Raidd gene, prior to causing overexpression of Raidd.

Cells in which overexpression of Raidd has been caused may advantageously be used in further procedures in which additional gene sequences are introduced into the cell. Methods in accordance with the present invention may therefore find application in the production of transgenic donor cells for use in nuclear transfer, in the production of transgenic embryonic stem cells or other progenitor cells (e.g. haematopoietic stem cells) prior to differentiation or determination of cell fate. Such modified stem cells may be useful in the treatment of disease conditions by means of transplantation of cells, tissues or organs ultimately derived from the modified stem cells.

Following modulation of the expression of Raidd in the cell, the cell may be subjected to additional genetic modification and/or manipulation. Subsequently, the cell may be allowed to continue the process of differentiation. This may be allowed to continue without intervention, or it may be the result of the presence of specific biochemical factors such as FGF, EGF, PDGF, IGF, ILGF, neurotrophic growth factor, GMCSF, LIF, which have the effect of causing the cell to differentiate into a desired cell type.

Examples of fully differentiated cell-types include but are not limited to: adipocytes, fibroblasts, myocytes (including cardiomyocytes), neurons, hepatocytes, lymphocytes, leukocytes, erythrocytes, pancreatic β-cells, enterocytes, oligodendrocytes, astrocytes, epithelial cells, alveolar cells, endothelial cells.

The control of differentiation according to a method of the present invention may also allow for a limited process of differentiation with a cell being arrested at a particular point along the developmental pathway to full differentiation. For example, an embryonic stem cell can give rise to a mesenchymal stem cell, which in turn can give rise to an osteoclast or an osteoblast cell, which finally reach terminal differentiation to become bone-forming cells. Enrichment and isolation of cells can take place at any point in this process according to a method of the invention. The terms “stem cell” and “progenitor cell” are used herein interchangeably unless the context specifies otherwise.

The present invention should be of utility in any animal cell or its descendants that passes through a process of differentiation. Animal cells may include those of any suitable animal species. For example, insects, amphibians, reptiles, birds, marsupials, and/or mammals.

It is anticipated that the invention may find particular utility in respect of human medicine. For example, stem cell-based therapies are actively contemplated for the treatment of Parkinson's disease, Huntingdon's disease, spinal cord injury, stroke, multiple sclerosis, diabetes, cardiac muscle repair, bone regeneration, joint repair (in the treatment of osteoarthritis).

Alternatively, the invention may find use in respect of non-human mammalian or avian species of agricultural importance, such as ungulates (including ovines, bovines, equines, porcines, caprines and the like), poultry (including chickens, turkeys and guinea fowl), rodents (including rats, mice and guinea pigs), and rabbits.

The term embryo (and by extension embryonic) in the context of the present invention is used to describe the developing animal following conception and the first division of the zygote until birth of the new-born animal, or the development of a nuclear transfer generated single-cell embryo until birth of a new-born animal. The term therefore includes more specialised descriptive terms “blastula”, “gastrula”, and “foetus” (or “fetus”). The term fetus can be used to describe an embryo when the first bone cells appear in the cartilage after implantation of the embryo. The term embryo includes “blastocyst” which describes the stage of development in humans at which implantation of the embryo into the uterine wall occurs in normal gestation which occurs when the inner cell mass (ICM) spreads inside the blastocoele as a flat disc). In other animals species, such as sheep and cattle, implantation may not occur until after the blastocyst stage and so the definition of “blastocyst” should be understood to extend to the stage of development which consists of inner cell mass cells and trophectoderm cells around a central blastocoele cavity.

Cattle and sheep embryos are routinely cultured to the blastocyst stage (after 5-7 days in embryo culture) prior to transfer into a suitable recipient animal while human embryos are generally transferred to the uterus on day 2 or 3 of development. However, culture of human embryos to the blastocyst stage is becoming more widely contemplated (Gardner, D. K. and Lane, M, Human Reproduction Update 3 367-382 (1997)) or at least up until development of the primitive streak. The primitive streak can be taken to have appeared in an embryo not later than the end of the period of 14 days beginning with the day the gametes are mixed, not counting any time during which the embryo is stored. In vitro embryo culture is required as part of many current and potential applications as well as for assisted reproduction in humans and other species. Many genetically-manipulated embryos produced by pronuclear injection have been cultured prior to transfer into a recipient, as have many embryos reconstructed by nuclear transfer. In vitro embryo culture has been used in protocols for the derivation of embryonic stem cells (ES cells) and ES cell-like cells and may be important in the development of cell-based therapies for clinical use.

The method may also find application to transgenic or genetically modified animals, at stages of development from embryos to adults, including chimeras and those prepared by nuclear transfer procedures or where the animal has been the subject of manipulation to alter its genetic content. Such methods may also find utility in cell-based transgenics using ES cells or other cells in culture using nuclear transfer, or in the generation of non-human mammals or other non-human animals of agricultural importance.

It should be noted that the term “transgenic”, in relation to animals, should not be taken to be limited to referring to animals containing in their genome or germ line one or more genes from another species, although many transgenic animals will contain such a gene or genes. Rather, the term refers more broadly to any animal whose germ line or genome has been the subject of technical intervention by recombinant DNA technology. So, for example, an animal in whose germ line an endogenous gene has been deleted, duplicated, activated or modified is a transgenic animal for the purposes of this invention as much as an animal to whose genome or germ line an exogenous DNA sequence has been added. In embodiments of the invention in which the animal is transgenic, the genetic modification may be undertaken using physical techniques such as microinjection into the male or female pronucleus of the zygote or into the cytoplasm or nucleus of an oocyte or embryo. Alternatively, the genetic modification can involve the use of mass transformation or transfection techniques such as electroporation, viral transfection (including the use of adenoviruses, retroviruses, adeno-associated means or synthetic retrotransposons), lipofection, microprojectile cell bombardment, antisense technology, vectors such as YAC and BAC or by using other means such as sperm. Furthermore the modification can benefit from intervention by homologous recombination, DNA repair mechanisms, including the use of restriction enzymes. Cell-mediated transgenesis can employ a variety of cells, including ES cells, EG cells and other stem cells or suitable cells from any mammalian species.

In embodiments of the invention in which the cell has been transfected with nucleic acid encoding a Raidd gene, the term transgenic should be understood as referring to another gene of interest that has been introduced into the cell in addition to the Raidd gene.

Where a heterologous gene sequence has been introduced into the cell (either Raidd or another gene of interest), available technologies such as cre-lox can be used to remove the gene from the cellular genome when no longer required.

Methods according to the present invention advantageously allow for improved isolation of populations of undifferentiated or relatively undifferentiated cells, for example stem cells or progenitor cells. This enables greater rates of enrichment and purification of stem cell or progenitor cell populations. This will have important implications for stem-cell based therapies of disease. It will also permit more genetic modification to be carried out if necessary in connection with stem cell populations prior to use. It is also conceived that methods in accordance with this invention will permit the isolation and characterisation of new stem cell or progenitor cell populations not previously known and/or recognised as such.

In a preferred embodiment of the invention, the method may comprise the steps of:

(1) isolating an undifferentiated cell from an embryo or an animal by means of

(i) extraction from subject in vivo; or

(ii) extraction from tissue or organ source in vitro; or

(iii) extraction from post-mortem subject

(2) transfecting a cell population derived from the undifferentiated cell isolated in (1) with a nucleic acid construct effective to modulate Raidd gene expression by overexpression

(3) culturing the cell population for a period of time sufficient to enrich the cell population; and optionally

(4) removing the nucleic acid construct from the cell population

In an alternative preferred embodiment of the invention, the method may comprise the steps of:

(1) generating a transgenic embryo or animal carrying a RAIDD gene introduced in a nucleic acid construct and expressing the Raidd gene in a selected lineage of cells

(2) isolating an undifferentiated cell from the transgenic embryo or animal by means of

(i) extraction from subject in vivo; or

(ii) extraction from tissue or organ source in vitro; or

(iii) extraction from post-mortem subject

(3) culturing a cell population derived from the undifferentiated cell isolated in (1) for a period of time sufficient to enrich cell the population; and optionally

(4) removing the nucleic acid construct from the cell population

According to step (3) in the schemes outlined above, the stem cell population may be expanded in culture, if required. The stem cell or progenitor cell population may then be subjected to further genetic modification such as the introduction of additional transgenes or nucleic acid constructs coding for a gene or genes of interest. The stem cell or progenitorcell population so obtained may be allowed to differentiate in culture without further intervention (other than excision of the RAIDD gene see below) or specific biochemical and/or factors may be introduced in order to direct differentiation to a desired cell phenotype. According to step (4) in the schemes outlined above, the elimination of RAIDD gene expression. could be accomplished by initially flanking the RAIDD gene sequences with lox P or frt sequence and at the appropriate time delivering Cre or frt protein, RNA or DNA sequence to the cells to catalyse the excision of the RAIDD sequences. Alternatively, the RAIDD sequence could be linked to an inducible or repressible regulatory system in such a manner that expression of the RAIDD gene could be reduced once the stem cell or progenitor population had been isolated and expanded, thus allowing it to continue further differentiation.

According to a second aspect of the invention there is provided the use of a nucleic acid sequence encoding Raidd in the control of differentiation of a cell.

According to a third aspect of the invention, there is provided a method of enriching a population of undifferentiated cells, the method comprising modulating expression of Raidd in the cells.

According to a fourth aspect of the invention, there is provided a method for preparing a population of stem cells, the method comprising

(i) isolating a stem cell in culture;

(ii) modulating expression of Raidd in the cell; and

(iii) allowing the stem cell population to expand in culture.

The stem cell population prepared according to this aspect of the invention may constitute a stem cell line. Methods in accordance with this aspect of the invention will permit screening of stem cell populations to identify previously unknown stem cells or sub-types of stem cells as differentiation will be arrested by means of modulation of the expression of Raidd.

According to a fifth aspect of the invention, there is provide a stem cell population or stem cell line prepared according to the fourth aspect of the invention.

Preferred features for the second and subsequent aspects of the invention are as for the first aspect mutatis mutandis.

In the description of the invention, reference is made to a number of drawings in which:

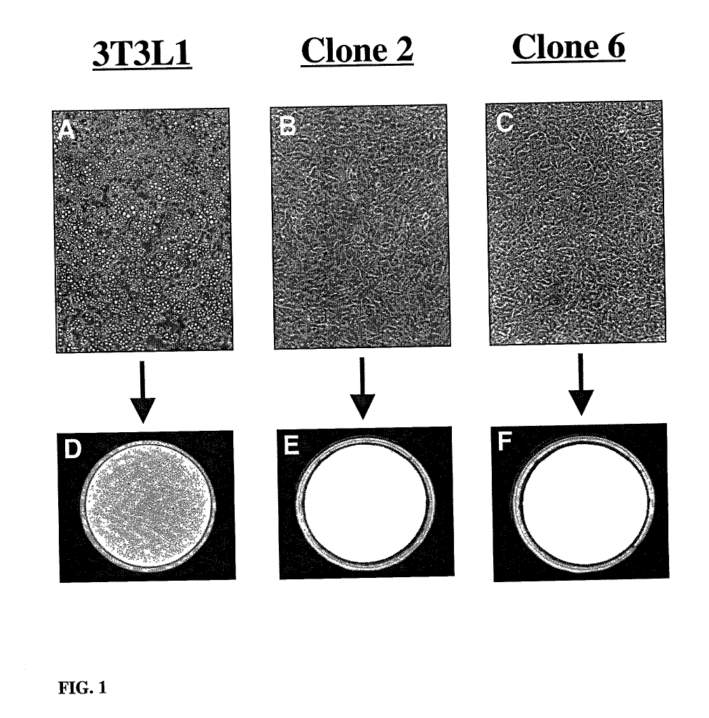

FIG. 1 shows differentiation of 3T3L1 cells into adipocytes upon hormonal induction and lack of differentiation in Raidd-transfected 3T3L1 cells. Panels A, B and C are photographs (100×magnification) of cells 8 days after hormonal induction to differentiation. Panel A shows parental 3T3L1 cells that differentiated normally after hormonal induction. In contrast, cells stably transfected with the murine Raidd cDNA did not undergo adipocyte differentiation in response to hormone treatment (Panels B and C for clones 2 and 6, respectively). Panels D, E and F show cells stained with Oil Red O, which stains specifically the lipids. Dark staining is observed in the parental 3T3L1 cells, (panel D) that accumulate triglycerides indicating appropriate differentiation into adipocytes. No staining is observed in the Raidd-transfected clones (panel E, F for clones 2 and 6, respectively) confirming the blockage of differentiation in these clones.

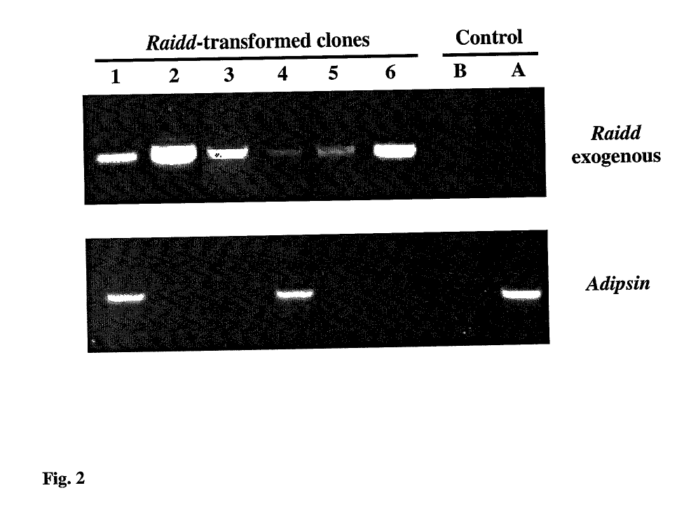

FIG. 2 shows Raidd expression from the introduced construct and adipsin expression, a marker for differentiation. 5 μg of total RNA from cells 8 days after hormonal induction was DNase treated, reverse transcribed with oligo (dT)12-18 mer (Gibco BRL) and subjected to PCR with primers (see Materials and Methods) specific for Raidd transcript from the introduced construct and adipsin. All six Raidd-transfected clones (1-6) express Raidd transcript from the construct. Adipsin, a marker of terminally differentiated adipocytes is expressed only in Raidd-transfected clones 1 and 4 that partially differentiated and in control cells 8 days after hormonal induction (A). No adipsin expression is detected in Raidd-clones exhibiting a complete blockage to differentiation (clones 2, 3, 5, 6) and in control cells before hormonal induction

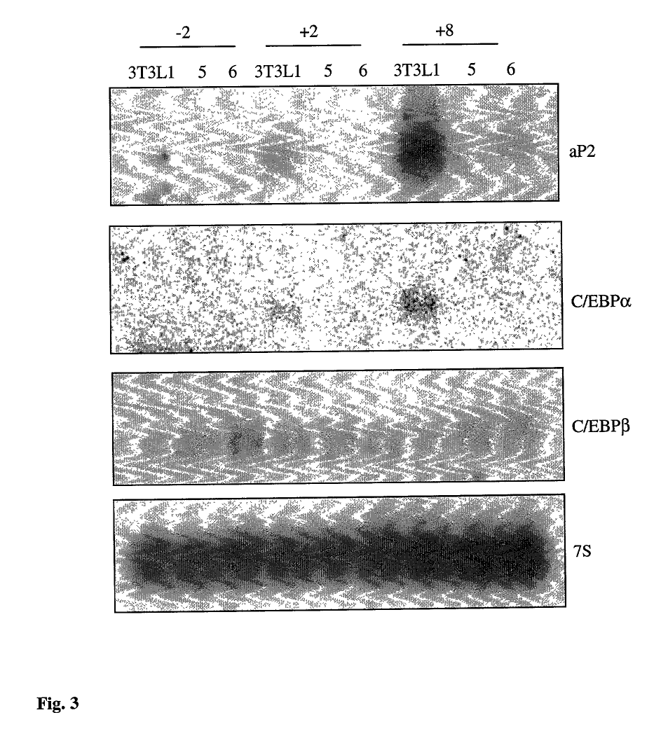

FIG. 3 shows Raidd overexpression inhibits terminal markers of the adipogenic program. 10 μg of total RNA from two Raidd-transfected clones and the parental cells 3T3L1 after different days of the differentiation program were run on a denaturing agarose gel and Northern blot hybridised with probes: aP2, C/EBPα, C/EBPβ, and 7S as loading control.

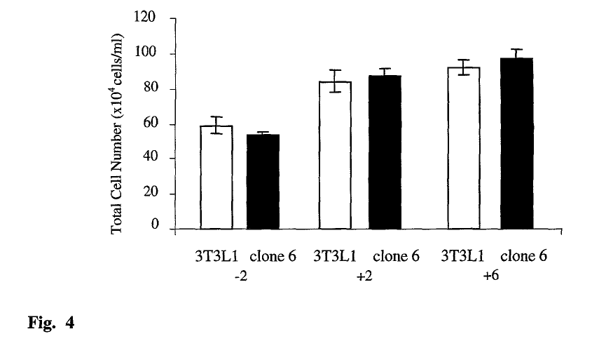

FIG. 4 shows cell number quantitation at different time points of the differentiation program. Cell number of the parental cells 3T3L1 (empty bar) and one of the clones transfected with Raidd (solid bar) were counted at different points of the differentiation program including at confluence (−2) after hormonal induction (+2) and after 6 days of differentiation (+6). Each bar represents the mean±SD of duplicates.

FIG. 5 shows the nucleotide sequence of the Raidd gene of the mouse the and presumed amino acid sequence of the RAIDD protein from Horvat, S, & Medrano, J. F. Genomics 54 (1) 159-164 (1998).

Reference is also made to the following Examples of the invention which are present for the purposes of illustration only and should not be construed as being limiting on the invention.

### EXAMPLES

A preadipocyte cell line (3T3L1) was stably transfected with a plasmid expressing the murine Raidd cDNA under the control of the adipocyte specific promoter aP2 (Ross et al Proc. Nat. Acad. Sci. USA 87 9590-4 (1990)). 3T3L1 is a preadipocyte cell line, initially described by Green and Meuth (Green, H & Meuth, M., Cell 2 127-33 (1974)), and later by many others, that differentiates in vitro and can exhibit most of the functions of adipocytes in vivo. Prior to differentiation, the preadipocyte cell line is morphologically similar to fibroblastic preadipose cells in the stroma of the adipose tissue. When appropriately induced with hormonal agents e.g. glucocorticoids, insulin-like growth factor-1, and cyclic AMP or factors that mimic these agents, (Student et al J. Biol. Chem. 255 4745-50 (1980)) committed preadipocytes differentiate into adipocytes in culture. When preadipocytes stably transfected with Raidd were induced to differentiate in the presence of hormones, most clones failed to differentiate. This effect was judged by the absence of lipid accumulation, a lack of expression of adipocytes-specific genes and a fibroblastic morphological appearance. In two of six clones, partial differentiation was observed, as judged by the partial accumulation of lipid and the death of differentiated adipocytes that escaped the initial blockage.

1. Materials and Methods

1.1 Plasmid Construct and Transfection Experiments

A 0.85 kb fragment containing the pA signal from SV40 was excised from the pA plasmid (kindly provided by Dr Wei Cui) by double digestion with Sma I/Not I, gel purified ligated to the aP2 promoter plasmid (kindly provided by Dr R Graves; Ross et al Proc. Nat. Acad. Sci. USA 87 9590-4 (1990)) that was also digested with Sma I/Not I. A 0.88 kb fragment corresponding to the murine Raidd cDNA was excised from the plasmid clone 6-1 (EMBL. No. AJ224738, Horvat. S, & Medrano, J. F., Genomics 54 159-64 (1998)) by double digestion with Sma I/Cla I, end-filled, and ligated into the unique Sma I site of the newly generated aP2-pA plasmid.

Stable transformation of 3T3L1 cells was carried out by calcium phosphate precipitation (Sambrook et al “Molecular cloning: A laboratory manual” (2^{nd} ed.) Cold Spring Harbour Laboratory Press, New York (1989)). Cells were transfected with a 5:1 molar ratio of chimeric construct/SV2 neo DNAs (15 μg per 10-cm dish). Selection of neomycin resistant foci was carried out with 400 μg/ml of neomycin for approximately 11 days, after which time individual clones were expanded in selective medium until analysis.

1.2 Cell Culture

Culture and differentiation of mouse embryo 3T3L1 preadipocytes (American Type Culture Collection) was performed as described previously (Student et al J. Biol. Chem. 255 4745-50 (1980)). Briefly, cells were propagated to confluence in medium DMEM with 10% FCS. On differentiation day 0, cells were fed with DMEM supplemented with 10% FCS, 0.5 mM methylisobutylxanthine, 10 μg/ml of insulin, and 1 μM dexamethasone 2 days later (differentiation day 2) and cultured in this medium for 6 days.

1.3 RNA Preparation and Northern Blot Analysis

Cell monolayers were washed twice with ice-cold phosphate buffered saline, and total RNA extracted with RNAzol B (Biogenesis, Poole, UK) following the manufacturer instructions. RNA was prepared at confluence (−2), after hormonal induction (+2) and after 6 days of differentiation (+6). Fifteen micrograms of total RNA was fractionated on 1.5% formaldehyde/agarose gels and blotted onto nylon membranes (Boehringer Mannheim). After UV cross-linking the filters were hybridised at 65° C. overnight in 0.5M sodium phosphate, 7% SDS with ³²P-labeled cDNA probes and washed in 2×SSC, 0.1% SDS for 10 minutes, followed by one wash of 15 minutes in 0.2×SSC, 0.1% SDS. The membranes were visualised using Phosphorimager (Molecular Dynamics) and AGFA x-ray film. cDNA probes for C/EBPα and C/EBPβ were obtained from the UK HGMP Resource Centre and aP2 cDNA probe was provided by Dr S Butterwith.

1.4 RT-PCR and QPCR Analysis

Total RNA from control and Raidd-transfected 3T3L1 cells was isolated 8 days after hormonal induction. 5 μg of total RNA (DNase-treated) was used to produce cDNA using the SuperScript® II RNase H Reverse transcriptase and the conditions given by the provider (Gibco BRL). The reverse transcription was performed at 42° C. for 50 minutes and heated at 70° C. for 15 minutes to inactivate the reverse transcriptase. 10% of this final reaction was used in a standard PCR reaction. The cDNA primers for Adipsin were forward (5′ ATGACGACTCTGTGCAGGTG 3′) and reverse (5′ GTATAGACGCCCGGCTTTTT 3′), whereas the cDNA primers for exogenous Raidd were forward (5′ AGCACCCTCCTGTGCAG 3′) that lies in the aP2 promoter and a reverse primer (5′ GCGAATGCACGTTGTGGGGA 3′) corresponding to an internal sequence of Raidd.

Relative quantitation of exogenous RAIDD mRNA and GAPDH mRNA was carried out using Real-Time Quantitative PCR (QPCR) (Higuchi et al Bio/Technology 11 1026-1030 (1993)).

RNA was subjected to Dnase 1 treatment and cDNA synthesized using the First Strand cDNA synthesis kit (Amersham Pharmacia Biotech). The ABI Prism 7700 Sequence Detector was used to monitor the accumulation of PCR product by measuring the increase in fluorescence resulting from the binding of SYBER Green to double-stranded DNA. The SYBER Green PCR Core Reagents kit (ABI, Warrington, Cheshire) including the internal reference dye, ROX, was used for all QPCR reactions.

1.5 Oil Red O Staining

Oil Red O staining was performed according to the method described by Green and Kehinde (Green, H. & Kehinde, O., Cell 1 113-116 (1974)). Cells were washed twice with PBS and fixed with 10% formalin in PBS for 15 minutes. After two washes in PBS, cells were stained for approximately 1 hour in freshly diluted Oil Red O solution (six parts Oil Red O stock solution in isopropanol). The stain was then removed and the cells washed twice with water and then photographed.

2. Results

2.1 Overexpression of Murine Raidd cDNA Blocks Differentiation of 3T3L1 Cells into Adipocytes.

3T3L1 cells can be induced to differentiate in the presence of agents that promote differentiation such as dexamethasone, insulin, methylisobutylxanthine (Student et al J. Biol. Chem. 255 4745-50 (1980)). Upon differentiation, the cells undergo a dramatic morphological change into adipocytes that results in a massive accumulation of cytosolic triglycerides by day 8 post induction (FIG. 1A). Two clones of 3T3L1 cells transfected only with the backbone plasmid containing the antibiotic resistance marker neomycin, differentiated as the 3T3L1 untransfected cells (data not shown). In marked contrast, four out of six 3T3L1 clones (clones 2, 3, 5, 6) stably transfected with the murine Raidd cDNA, failed to differentiate and did not show accumulation of cytosolic triglycerides when treated under identical conditions as the parental cells (two clones shown in FIG. 1B, C). Upon examination by Oil Red O staining, which stains specifically the lipid droplets (Green, H. & Kehinde, O., Cell 1 113-116 (1974)), parental 3T3L1 cells exhibited strong staining for lipid droplets (FIG. 1D) whereas the Raidd-transfected clones showed no staining (FIG. 1E, F). Similar results were obtained in a repeated transfection experiment. No staining for lipid droplets was observed in the Raidd-transfected 3T3L1 cells even 2 weeks post-induction or when the induction protocol was initiated 4 or 6 days post-confluence, instead of 2 days as recommended in the published differentiation protocol (Student et al J. Biol. Chem. 255 4745-50 (1980)).

In two of six Raidd-transfected clones (clones 1, 4) we observed partial differentiation into adipocytes in that some morphological change from fibroblast-like to rounded up cells was detected. One of these clones (clone 1) had shown reduced accumulation of cytoplasmic triglycerides whereas in the other clone (clone 4) partial differentiation into adipocyte occurred but was followed by pronounced cell death. Overall, four of six clones showed a complete blockage of differentiation with the other two exhibiting only partial differentiation. It was concluded that transformation of 3T3L1 cells with the murine Raidd cDNA results in the block of differentiation into adipocytes upon induction of the differentiation programme.

2.2 Expression Analysis of Exogenous Raidd mRNA and Adipsin, a Marker of Terminally Differentiated Adipocytes

Expression of exogenous Raidd was assessed by RT-PCR and by real-time quantitative polymerase chain reaction analysis (QPCR). For RT-PCR, total RNA of the six Raidd-clones after 8 days of the induction to differentiation was used as a template. In order to detect specifically the expression of the exogenous Raidd, a pair of primers was designed to amplify a region containing aP2 promoter (5′ primer) and a reverse primer corresponding to an internal sequence of Raidd (3′ primer, see Materials and Methods). All transfected clones expressed the Raidd mRNA from the integrated construct (clones 1-6, FIG. 2), whereas, as expected, control parental cells before (FIG. 2, lane B) or after differentiation into adipocytes (lane A) showed no signal for the exogenous Raidd.

For the real-time quantitative polymerase chain reaction analysis, the same primers for exogenous Raidd expression as for RT-PCR were used (see above). Expression of exogenous Raidd was assessed relative to Gapdh expression in clones 5 and 6 that showed a complete blockage to differentiation and control 3T3L1 cells. Analyses were carried out on cells at both 2 days prior to induction to differentiate (−2) and at 8 days post-induction (+8). Expression of exogenous Raidd was evident in clones 5 and 6 both before and after induction, but undetectable in control samples. For individual clones, expression of exogenous Raidd relative to Gapdh did not change significantly as a result of the induction process, however, expression in clone 6 was approximately 100 fold greater than that observed in clone 5 (clone 5:−2=3×10⁻⁵, +8=3.5×10⁻⁵; clone 6:−2=6×10⁻³, +8=1.2×10⁻²). These results confirm the RT-PCR analysis (FIG. 2) that also showed a higher intensity of the amplified product in clone 6 suggesting a higher expression of exogenous Raidd in this clone.

Next we examined if the block of differentiation in Raidd clones is associated with the lack of expression of the adipsin in cells 8 days after hormonal induction. Adipsin is a serine protease whose cDNA was isolated by differential screening of preconfluent and differentiated 3T3L1 cells (Cook et al Science 237 402-5 (1987)). It is found in vivo in the circulation of both animals and humans and is produced and secreted in vivo by differentiated 3T3L1 cells, thus making this gene a marker for terminally differentiated adipocytes. RT-PCR for adipsin produced a signal only in the two clones that partially differentiated into adipocytes (clones 1 and 4, FIG. 2) and, as expected, in the control parental cells (3T3L1) after differentiation into adipocytes (lane A, FIG. 2). In contrast, no induction of the expression of this gene was observed for the clones 2, 3, 5 and 6, thus confirming a complete block in the differentiation of these cells as observed in the morphological and Oil Red staining analysis (FIG. 1).

2.3 Raidd Overexpression Inhibits Expression of Various Markers During Adipogenesis

Adipocyte growth and differentiation follow patterns of sequential B-ZIP protein expression, including AP-1 (Distel et al Cell 49 835-844 (1987)) and C-EBP family proteins (McKnight et al Genes & Dev. 3 2021-2024 (1989)). To examine if this pattern of sequential expression is affected in the Raidd-transfected 3T3L1 clones, we conducted Northern analysis of the transcription factors involved in the differentiation of 3T3L1 into adipocytes. RNA from two Raidd-transfected clones (clones 5, 6, FIG. 3) and parental cells isolated before hormonal induction and 2 and 8 days after induction was analysed.

In the control cells, expression of the mRNA encoding fatty acid binding-protein aP2 (Bernlohr et al Proc. Natl. Acad. Sci. USA 81 5468-72 (1984)) was first detectable at low levels after the induction to differentiate (day 2) and rose to maximal levels by day 8. Control cells differentiated normally and accumulated triglyceride droplets in the cytoplasm of the cells (not shown). In the Raidd-transfected clones, failure to differentiate was observed again and aP2 expression could not be detected in clone 5 whereas in clone 6 a very low signal after 8 days of hormonal induction was observed. This Northern blot was rehybridised with the C/EBPα and C/EBPβ cDNA probes. Consistent with earlier studies done in 3T3L1 cells (Birkenmeier et al Genes Dev. 3 1146-56 (1989)), C/EBPα transcripts was not detected prior to the differentiation and was first detected at low levels on day 2 and increased at day 8 in the control cells. However, no signal was detected in the two Raidd-transfected clones. The mRNA encoding for C/EBPβ that is expected to be expressed in undifferentiated 3T3L1 cells, on the other hand, was detectable in both control and Raidd-transfected cells and remained detectable during the later half of the differentiation program consistent with earlier studies (Cao et al Genes Dev. 5 1538-52 (1991)). These findings show that the blockage in the differentiation of the Raidd-transfected clones correlate with the lack of sequential expression of transcription factors during adipocyte differentiation such as C/EBPα and aP2 .

2.4 Cell Proliferation is not Impaired in Raidd-transfected Clones

To assess if cell division was impaired in the Raidd-transfected cells, proliferation rate of the cells was examined at different stages of growth before and after induction with hormones. Wild type 3T3L1 cell and Raidd-transfected cells (clone 6) were plated with the same initial numbers on 6 well dishes and cells were counted at confluence (−2), after induction with the hormones (+2) and after 6 days of hormonal induction. FIG. 4 shows that the number of cells of 3T3L1 cells and Raidd-transfected clone at confluence (−2) is essentially the same indicating that overexpression of Raidd does not affect proliferation rate in the preadipocyte stage. After hormonal induction (FIG. 4, +2, +6) both groups increased the number of cells due to the clonal expansion induced by the combination of mitogenic and adipogenic signals provided in the differentiation medium. However, the number of cells was similar in both groups although parental 3T3L1 cells underwent differentiation into adipocytes whereas Raidd-transfected clone remained at the preadipocyte stage. These results show that cell proliferation is not impaired in the Raidd-transfected clone either before or after hormonal induction suggesting that inhibition of cell division does not cause the blockage to differentiation of Raidd-transfected clones.

3 Discussion

Transfection of 3T3L1 cell with the murine Raidd cDNA resulted in a complete blockage to differentiation into adipocytes in four of six clones, after exposure to the cocktail of hormones/inducers. In two of six clones, partial differentiation was observed with one clone showing a partial accumulation of triglycerides and the other showing a pronounced cell death of the partially differentiated adipocytes. It is possible that in the two partially differentiated Raidd-transfected clones either the timing and/or the level of exogenous Raidd expression was different from the 4 completely blocked clones such that it allowed the cells to partially differentiate. Even in these two clones differentiation was not normal and did not proceed to completion. All of the Raidd-transfected clones showed either blocked or impaired differentiation. Therefore we demonstrate that stable transformation of 3T3L1 cells with Raidd has an inhibitory effect on differentiation into adipocytes.

These findings show that overexpression of Raidd blocks the differentiation of 3T3L1 cells into adipocytes. The Raidd cDNA used was under the control of aP2 promoter (Hunt et al Proc. Natl. Acad. Sci. USA 83 3786-90 (1986)). aP2 promoter does not support expression in preadipocytes and its maximum activity is reached after ˜6 days of the differentiation program. However, Lin and Lane (Lin, F. T. & Lane, M. D., Genes Dev 6 533-44 (1992)) have observed a low level of aP2 mRNA by RNase protection analysis in confluent 3T3L1 preadipocytes and we too have observed expression of a fluorescent reporter gene (GFP) driven by the aP2 promoter in preadipocytes (Felmer R. unpublished observations). Therefore, a low level of expression from this promoter can be observed earlier in the adipogenic program even before transactivation from C/EBPα. Our real time quantitative PCR analysis also showed that the aP2 promoter in our transgene was already active in early preadipocytes. With QPCR we found that exogenous Raidd was expressed at the same level before and 8 days after induction in the two Raidd-transfected clones that did not differentiate. It is possible that this sustained expression of exogenous Raidd starting from early stage preadipocytes rendered the cells susceptible to the Raidd-mediated inhibition of differentiation.

Overexpression of Raidd could block cell differentiation by perturbing one or several steps that are important for the terminally differentiated state. Raidd overexpression could be acting in stimulating the expression of anti-differentiation genes or alternatively inhibiting the expression of pro-differentiation genes. By analysing patterns of gene expression characteristic of each phase of the differentiation program, it was determine that the Raidd-mediated block in the differentiation of 3T3L1 cells seems to be manifested during the terminal stages of the differentiation in clones that failed to differentiate. Expression of a very early differentiation marker C/EBPβ was no altered in the Raidd clones whereas the expression of differentiation marker C/EBPα expressed during adipogenesis and terminal stage-specific markers aP2 and adipsin was absent or decreased.

Inhibition of the differentiation of 3T3L1 was demonstrated in other studies by antisense C/EBPα technique (Lin, F. T. & Lane, M. D., Genes Dev 6 533-44 (1992)) that blocked the expression of C/EBPα. This in turn inhibited expression of aP2, SCD1 and GLUT4 and the accumulation of cytoplasmic triglycerides (Lin, F. T. & Lane, M. D., Genes Dev 6 533-44 (1992)), which confirmed that C/EBPα is required for preadipocyte differentiation. Induction of C/EBPβ and C/EBPδ expression is thought to be necessary for the subsequent induction of C/EBPα and PPARγ, which then act to co-ordinate the expression of a number of fat specific genes required for lipid metabolism, including aP2, SCD1 and Glut4. In our Raidd clones that did not differentiate C/EBPβ expression was not altered but the expression of C/EBPα was undetectable. We can thus speculate that Raidd overexpression causes down regulation of prodifferentiation proteins such as C/EBPα and possibly PPARγ (not analysed) preventing transactivation of the downstream targets in the differentiation pathways of adipocytes.

Our study demonstrated that overexpression of Raidd represses adipogenesis indicating that Raidd may function, directly or indirectly, in differentiation. It would, therefore, be of interest to test in future if lack of Raidd function can promote preadipocyte differentiation to confirm our current speculation that Raidd functions as a suppressor of adipocyte differentiation. Raidd contains an amino-terminal CARD (Caspase Recruitment Domain) region and a carboxy-terminal “death domain” (Duan, H. & Dixit, V. M., Nature 385 86-9 (1997)) and has been so far only been implicated to act as an adapter protein in the apoptotic signalling pathway. Our results suggest that Raidd does not function only in apoptosis but could also be involved in differentiation.

Several studies have shown that the inhibition of cell division of cultured preadipocyte clearly blocks their subsequent differentiation (Timchenko et al Genes Dev. 10 804-15 (1996)). Nevertheless, we found no evidence of impaired cell division in these clones transfected with Raidd, since analysis of the proliferation rate of Raidd-transfected clone throughout the different steps of the differentiation program showed no significant difference with the control cells. Furthermore, blocked cells proliferated normally even after confluent dishes that had been treated with the hormones were plated out at lower densities suggesting that apoptosis was not triggered in these blocked clones.

In summary, Raidd overexpression inhibits 3T3L1 preadipocyte differentiation as demonstrated by cell morphology, lipid accumulation, and expression of adipocytespecific markers. The differentiation process in Raidd-transfected clones is associated with inhibition of expression of differentiation factor C/EBPα and late-stage differentiation markers aP2 and adipsin. Further studies need to shed light on the mechanism of how Raidd overexpression induces blockage to preadipocyte differentiation and to characterise a role of Raidd gene in differentiation in preadipocytes or other cell types.

1. A method of controlling the differentiation of a cell, the method comprising modulating the expression of Raidd in the cell.

2. A method as claimed in claim 1, in which modulation of expression is overexpression of Raidd in the cell.

3. A method as claimed in claim 2, in which overexpression of Raidd is effected by an inducible promoter.

4. A method as claimed in claim 2, in which overexpression of Raidd is effected by a lineage specific promoter.

5. A method as claimed in claim 1, in which the cell is subsequently allowed to differentiate.

6. A method as claimed in claim 5, in which the cell subsequently differentiates to become an adipocyte, fibroblast, myocyte (including a cardiomyocyte), neuron, hepatocyte, lymphocyte, leukocyte, erythrocyte, pancreatic β-cells, enterocytes, oligodendrocytes, astrocytes, epithelial cells, alveolar cells, endothelial cells.

7. The use of a nucleic acid sequence encoding Raidd in the control of differentiation of a cell.

8. A method of enriching a population of undifferentiated cells, the method comprising modulating expression of Raidd in the cells.

9. A method for preparing a population of stem cells, the method comprising
(i) isolating a stem cell in culture;
(ii) modulating expression off Raidd in the cell; and
(iii) allowing the stem cell population to expand in culture.

10. A stem cell population or stem cell line prepared according to a method of claim 9.
